(19) United States
(12) Patent Application Publication
(10) Pub. No.: US 2016/0216222 A1
(43) Pub. Date: Jul. 28, 2016

(54) Sensor for Detecting a Gas

(71) Applicant: CONTINENTAL AUTOMOTIVE GMBH, Hannover (DE)

(72) Inventors: Johannes Ante, Regensburg (DE); Torsten Reitmeier, Wackersdorf (DE); Andreas Wildgen, Nittendorf (DE)

(73) Assignee: Continental Automotive GmbH, Hannover (DE)

(21) Appl. No.: 15/025,402

(22) PCT Filed: Sep. 26, 2014
(86) PCT No.: PCT/EP2014/070589

§ 371 Date: Mar. 28, 2016

(30) Foreign Application Priority Data
Sep. 27, 2013 (DE) 10 2013 219 531.1

(51) Int. Cl. G01N 27/14; G01N 33/00

(52) U.S. Cl. CPC G01N 27/14; G01N 33/0036

(57) ABSTRACT

A sensor for detecting a gas may include a transport layer for transporting ions, a first electrode and a second electrode spaced apart from one another by the transport layer, a heating device controlled by a voltage source, and a second voltage source applying a voltage difference across the electrodes. The transport layer may be conductive for the ions starting from a specific temperature. As a result of applying the voltage difference, ions stream from the first electrode through the transport layer to the second electrode if the first and second electrodes are in contact with the gas. The voltage source may provide a voltage potential of zero volts averaged over time or provide the voltage potential of the first electrode at the heating device.

### CROSS-REFERENCE TO RELATED APPLICATIONS

This application is a U.S. National Stage Application of International Application No. PCT/EP2014/070589 filed Sep. 26, 2014, which designates the United States of America, and claims priority to DE Application No. 10 2013 219 531.1 filed Sep. 27, 2013, the contents of which are hereby incorporated by reference in their entirety

### TECHNICAL FIELD

The present disclosure relates to a sensor for detecting a gas, in particular an oxygen sensor for detecting an oxygen content.

### BACKGROUND

A sensor for detecting a gas, in particular oxygen, in the environment of the sensor may include a transport layer for transporting ions. The sensor can have, for example, a transport layer composed of yttrium-doped zirconium oxide which is conductive for ions, for example for oxygen ions, starting from a specific temperature. Furthermore, the sensor can comprise electrodes which are arranged at the transport layer and are electrically insulated from one another by the transport layer. The electrodes can be constructed from porous platinum. As a result of the application of a suitable voltage difference between the electrodes, one of the electrodes can be operated as a cathode and the other electrode as an anode. In order to heat the transport layer, a heating device can be provided which is separated from the transport layer by a protective layer.

If the sensor is embodied as an oxygen sensor, the arrangement can be regulated in such a way that the oxygen concentration in the region of the cathode is approximately zero. If the arrangement is introduced into an oxygen-containing environment, with the result that the electrodes come into contact with an oxygen-containing gas or environment, oxygen molecules diffuse out of the environment to the cathode. At the cathode which is formed from porous platinum, the oxygen molecules are converted into oxygen ions. Owing to the voltage difference which is present between the cathode and the anode, the oxygen ions migrate from the cathode to the anode. If the anode is also constructed from porous platinum, the oxygen ions recombine at the anode to form oxygen molecules which are output to the environment again.

Information about the oxygen content in a measurement gas in the environment of the sensor can be acquired by measuring the current which occurs when the voltage difference is applied between the cathode and the anode. The measured current is dependent on the concentration or the partial pressure of the oxygen in the measurement gas.

In order to operate the heating device, a control voltage is applied to the heating device. The temperature of the heating device can be regulated as a function of the applied control voltage. In such a sensor, a clear drift of the measurement current as a function of the heating power of the heating device becomes apparent.

### SUMMARY OF THE INVENTION

Some embodiments of the present disclosure may include a sensor for detecting a gas in an environment of the sensor using a current which is measured between the electrodes of the sensor. Such embodiments depend virtually exclusively on the composition of the gas in the environment of the sensor and are very largely independent of the heating power of a heating device.

Some embodiments of the sensor comprise a transport layer for transporting ions, wherein the transport layer is conductive for the ions starting from a temperature. Furthermore, the sensor has a first electrode and a second electrode which are arranged spaced apart from one another and are separated from one another by the transport layer. Furthermore, the sensor comprises a heating device for heating the transport layer to the temperature starting from which the transport layer becomes conductive. Furthermore, the sensor has a controllable power source or voltage source for generating a control voltage for controlling a temperature of the heating device. The power source or voltage source is connected to the heating device. The sensor can have a further controllable power source or voltage source for applying a first potential to the first electrode and for applying a second potential, different from the first potential, to the second electrode. When the first potential is applied to the first electrode, and when the second potential is applied to the second electrode, a stream of ions occurs from the first electrode through the transport layer to the second electrode if the first and second electrodes are in contact with the gas. The controllable power source or voltage source can be controlled in such a way that a voltage potential of 0 V averaged over time or the voltage potential of the first electrode is present at the heating device.

In order to pump oxygen ions, the voltage difference between the first and second electrodes is generated in such a way that the first electrode is operated as a cathode and the second electrode as an anode. If the controllable power source or voltage source for generating the control voltage for the heating device generates the control voltage in such a way that the average voltage or the average voltage potential at the heating device is higher than the voltage/the voltage potential at the anode, the ions, in particular oxygen ions, exhibit the tendency to migrate to the higher voltage or to the higher voltage potential of the heating device instead of to move to the anode, and to re-enter the environment of the gas from there.

In order to prevent the ions being able to migrate from the cathode directly to the heating device, a protective layer can be provided between the transport layer and the heating device. The protective layer is embodied in such a way that it has, for transportation of the ions, a higher resistance than the transport layer. The protective layer can, for example, be unpassable for oxygen ions. Owing to the tendency of the ions to migrate to the higher voltage potential of the heating device, the ions can accumulate on the protective layer and choke off a conductive channel of the transport layer between the cathode and the anode.

In some embodiments, the controllable power source or voltage source for generating the control voltage for the heating device generates an average voltage potential of 0 V at the heating device. This prevents the ions in the transport layer from being attracted by a positive voltage potential of the heating device, which voltage potential is higher than the voltage potential at the anode.

In some embodiments, the controllable power source or voltage source for controlling the heating device can generate the control voltage in such a way that the voltage potential which is generated at the heating device corresponds to the voltage potential of the first electrode, that is to say to the voltage potential of the cathode. Since the voltage potential of the second electrode, that is to say the anode, is higher than the voltage potential of the first electrode, that is to say the cathode, during their migration through the transport layer the ions are not affected by the voltage potential of the heating device, which voltage potential corresponds, according to the alternative embodiment, to the voltage potential of the cathode. Therefore, in the alternative second embodiment of the sensor, it can also be ensured that the stream of ions within the transport layer is virtually independent of the power set at the heating device.

### BRIEF DESCRIPTION OF THE DRAWINGS

The invention will be explained in more detail below with reference to figures which show exemplary embodiments of the present invention.

FIG. 1 shows an embodiment of a sensor for detecting a gas in an environment of the sensor, and

FIG. 2 shows an embodiment of a controllable power source or voltage source for generating a control voltage for controlling a temperature of the heating device.

### DETAILED DESCRIPTION

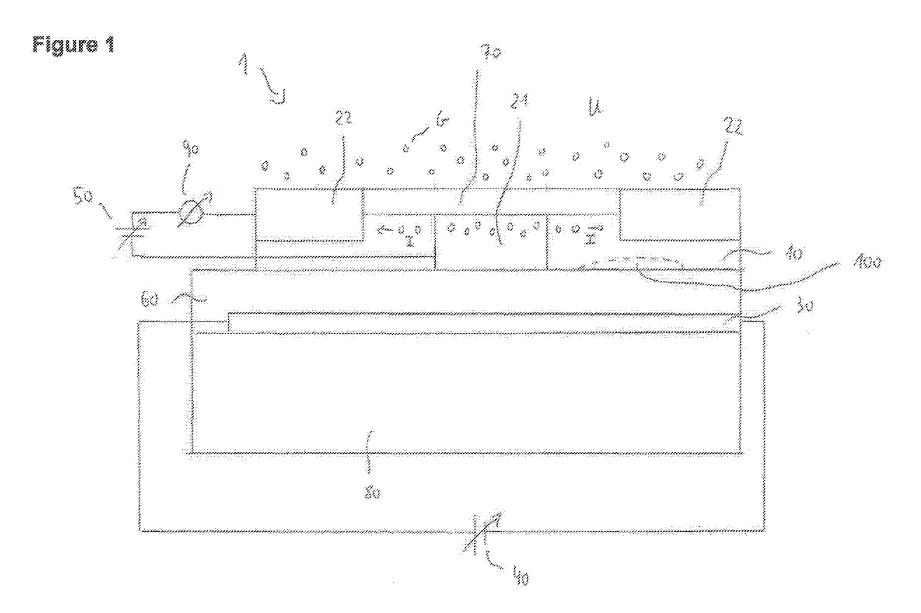

FIG. 1 shows a sensor 1 for detecting a gas G in an environment U of the sensor. The sensor may detect an oxygen content in an environment of the sensor. The sensor comprises a transport layer for transporting ions I, in particular oxygen ions. The transport layer 10 is conductive for the ions I starting from a specific temperature. The transport layer 10 can contain, for example, yttrium-doped zirconium oxide. A first electrode 21 and a second electrode 22 are arranged in the transport layer 10.

In order to heat the transport layer 10 to the temperature starting from which the transport layer 10 becomes conductive for the ions I, a heating device 30 is provided. The heating device 30 can be embodied as a heating wire, for example as a platinum coil. The heating power of the heating device 30 can be regulated by applying a control voltage. The sensor 1 has for this purpose a controllable power source or voltage source 40 for generating a control voltage for controlling a temperature of the heating device 30. The power source or voltage source 40 is connected to the heating device 30.

A controllable power source or voltage source 50 is provided for the application of a voltage difference between the first electrode 21 and the second electrode 22. The controllable power source or voltage source 50 is embodied in such a way that a voltage difference can be generated between the first and second electrodes 21, 22 in such a way that the first electrode 21 can be operated as a cathode, and the second electrode 22 as an anode.

A protective layer 60 is provided between the transport layer 10 and the heating device 30. The protective layer 60 is designed to have a higher resistance for transportation of ions I than the transport layer 10. The protective layer 10 can be embodied, for example, as an aluminum oxide layer through which oxygen ions cannot migrate. A diffusion barrier layer 70 can be arranged between the electrode 21 and an environment U in which the gas G is present. The first electrode 21 is therefore arranged between the diffusion barrier layer 70 and the protective layer 60 and is embedded in the transport layer 10. The heating device 30 is arranged on a substrate 80.

The sensor which is illustrated in FIG. 1 can be arranged, for example, for measuring an oxygen concentration in the intake section of an exhaust gas recirculation system. In such an application, the oxygen content in the gas G can be between 10% and 21% after the exhaust gas recirculation system opens into the environment U, for example. The sensor is regulated in such a way that an oxygen concentration in the region of the cathode 21 is approximately zero. Oxygen molecules of the gas G therefore diffuse from the environment U through the diffusion barrier layer 70 to the cathode 21. At the cathode 21 which is constructed from porous platinum, an oxygen molecule takes up four electrons and therefore becomes an oxygen ion.

Owing to the doping of the transport oxide layer, for example of a layer composed of zirconium oxide which can be doped with 8% yttrium oxide, fault points which permit diffusion of oxygen ions come about in the lattice of the transport layer 10. If the controllable power source or voltage source 50 applies a voltage difference to the electrode 21 and to the electrode 22 in such a way that the electrode 21 is operated as a cathode and the electrode 22 as an anode, the negatively charged ions I are attracted by the anode 22. They migrate to the anode 22, ideally through the ion-conductive transport layer 10, which is heated by the heating device 10 to a specific temperature, for example to a temperature of more than 650° C. The ions recombine in the anode formed from porous platinum and are output again into the environment U of the gas G as oxygen molecules.

The stream of ions through the transport layer 10 is higher the higher the oxygen content or the oxygen partial pressure in the gas G. In order to measure the stream of ions, which is a measure of the oxygen content in the gas G, an ammeter 90 can be arranged in the circuit between the controllable power source or voltage source 50 and the electrodes 21, 22.

It becomes apparent that in the case of a sensor arrangement in which the controllable power source or voltage source 40 generates a control voltage for the heating device 30 in such a way that the average voltage at the heating device 30 is higher than the voltage potential at the anode 22, a significant drift of the measurement current occurs. The measurement current which is detected with the ammeter 90 is therefore dependent not only on the oxygen concentration in the gas G but also on the heating power which is set or the voltage potential which is applied to the heating device 30 in relation to the anode 22.

If, for example, the controllable power source or voltage source 50 applies a voltage potential of 2.1 V to the cathode 21 and a voltage potential of 2.5 V to the anode 22, and the controllable power source or voltage source 40 applies an average voltage potential between 6 V and 11 V to the heating device 30, the oxygen ions I are attracted more strongly by the heating device 30 than by the anode 22 despite the protective layer 60. Although the protective layer 60 prevents a direct stream of ions to the heating device 30—apart from a small stream of ions owing to fault points, oxygen reservoirs 100 build up above the protective layer 60 and impede the pumping of oxygen ions toward the anode 22.

As a result, the force of the ion pump is reduced. The very high electrical field strength between the heating device and the actual pump cell substantially brings about the destruction or damage to the Y—ZrO₂ structure of the transport layer 10, as a result of which it is permanently damaged. Both effects ultimately bring about a decrease in the pumping current or measurement current and therefore a drift of the output signal.

In some embodiments, the voltage potential at the heating device 30 is set by the controllable power source and voltage source 40 in such a way that a preferred direction of the oxygen ion movement to the anode 22 is ensured and a pumping effect of the heating device 30 is prevented. The controllable power source or voltage source 40 can actuate the heating device 30 for this purpose in such a way that a potential of 0 V which is averaged over time is present at the heating device 30. The controllable power source or voltage source 40 can be designed, for example, to generate an alternating voltage. The controllable power source or voltage source 40 can be designed, in particular, to generate the alternating voltage in such a way that a positive and negative potential with the same level are present at the heating device 30 alternately. As a result, an average potential of 0 V occurs at the heating device 30. The controllable power source or voltage source 40 can generate, for example, a pulse-width-modulated voltage as the control voltage.

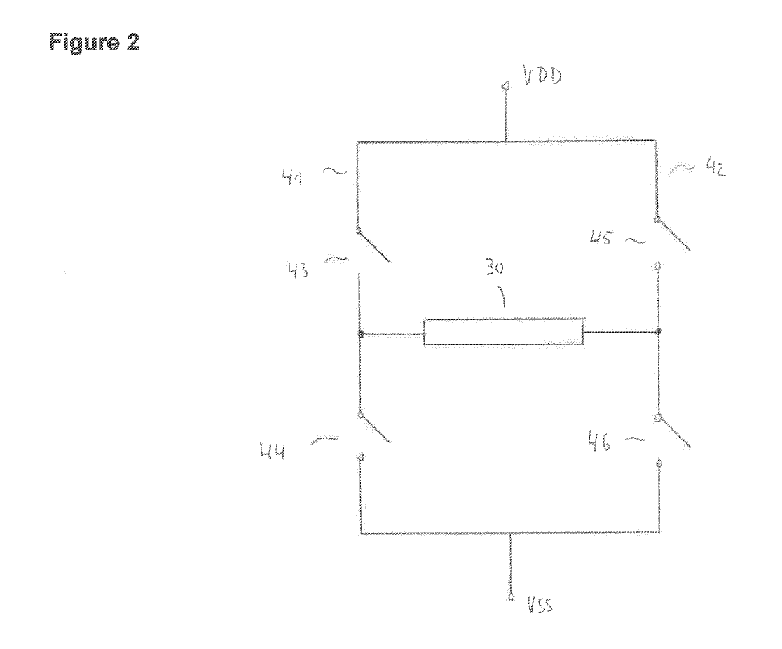

FIG. 2 shows a possible embodiment of the controllable power source or voltage source 40 as a full-bridge circuit, in particular as an H full-bridge circuit. The full-bridge circuit has a current path 41 and a current path 42 which are connected between a positive supply potential VDD and a negative supply potential VSS. Controllable switches 43 and 44 are arranged in the current path 41. Controllable switches 45 and 46 are arranged in the current path 42. The controllable switches may be embodied, for example, as transistors. A first side of the heating device 30 is connected to the current path 41, and a second side of the heating device 30 is connected to the current path 42. The first side of the heating device 30 is connected between the controllable switches 43 and 44 to the current path 41. The second side of the heating device 30 is connected to the current path 42 between the controllable switch 45 and the controllable switch 46.

By means of the full-bridge circuit it is possible to apply alternately a positive and negative voltage potential with the same level to the heating device 30 with respect to one side of the heating device. In order to generate a positive voltage potential, for example the controllable switches 43 and 46 are switched on and the controllable switches 44 and 45 are switched off. In order to apply a negative voltage potential with the same level, the controllable switches 43 and 46 are subsequently switched off and the controllable switches 44 and 45 are switched on.

In some embodiments, it is possible to prevent oxygen ions being attracted by a voltage potential of the heating device 30 which is higher than the voltage potential at the anode. In such embodiments of the sensor, the controllable power source or voltage source 40 can generate the control voltage for the heating device 30 in such a way that the voltage potential of the cathode 21 is present at the heating device 30. The anode 22 therefore has the highest voltage potential of the arrangement, with the result that the ions are attracted from the cathode to the anode. With both of the aforesaid embodiments of the sensor it is possible to ensure that the highest electrical voltage potential is provided at the anode 22, to where the oxygen ions are pumped according to normal use.

What is claimed is:

1. A sensor for detecting a gas in an environment of the sensor, the sensor comprising:
a transport layer for transporting ions, the transport layer conductive for the ions starting from a specific temperature,
a first electrode and a second electrode spaced apart from one another by the transport layer,
a heating device for heating the transport layer to the specific temperature,
a controllable voltage source generating a control voltage for controlling the heating device connected to the heating device,
a second controllable voltage source applying a voltage difference between the first and second electrodes,
wherein as a result of applying the voltage difference between the first electrode and the second electrode, a stream of ions occurs from the first electrode through the transport layer to the second electrode if the first and second electrodes are in contact with the gas,
wherein the controllable voltage source is controlled in such a way that a voltage potential of zero volts averaged over time or the voltage potential of the first electrode is present at the heating device.

2. The sensor as claimed in claim 1, wherein the controllable voltage source generates an alternating voltage.

3. The sensor as claimed in claim 2, wherein the controllable voltage source generates the alternating voltage in such a way that a positive and negative voltage potential with the same level is alternately present at the heating device.

4. The sensor as claimed in claim 3, wherein the controllable voltage source generates a pulse-width-modulated voltage.

5. The sensor as claimed in claim 1, wherein the controllable voltage source includes an H full-bridge circuit.

6. The sensor as claimed in claim 1, wherein the second controllable voltage source generates the voltage difference between the first and second electrodes in such a way that the first electrode can be operated as a cathode, and the second electrode as an anode.

7. The sensor as claimed in claim 1, further comprising
a protective layer which has a higher resistance to the transport of ions than the transport layer,
wherein the protective layer separates the transport layer from the heating device.

8. The sensor as claimed claim 1,
wherein:
the heating device includes a heating wire, and
the transport layer contains yttrium-doped zirconium oxide.

9. The sensor as claimed in claim 1, further comprising
a diffusion barrier layer,
wherein the first electrode is arranged between the diffusion barrier layer and the protective layer and is embedded in the transport layer.

10. The sensor as claimed in claim 1,
wherein the sensor senses oxygen and
the transport layer transports oxygen ions.

11. A method for sensing a gas, the method including the steps of:
heating a transport layer to a specific temperature at which the transport layer becomes conductive for ions of the gas with a heating device,
applying a voltage differential across a first electrode and a second electrode separated by the transport layer,
controlling a power source to provide a voltage potential of zero volts averaged over time or the voltage potential of the first electrode at the heating device, and
measuring the current between the first and the second electrode.

12. The method as claimed in claim 11, wherein the voltage differential comprises an alternating voltage.

13. The method as claimed in claim 12, wherein the the alternating voltage provides an alternate positive and negative voltage potential with the same level at the heating device.

14. The method as claimed in claim 13, wherein the alternating voltage includes a pulse-width-modulated voltage.

15. The method as claimed in claim 1, wherein the gas comprises oxygen and the transport layer transports oxygen ions.

16. A sensor for measuring an oxygen concentration in the intake section of an exhaust gas recirculation system, the sensor comprising:
a transport layer for transporting oxygen ions, the transport layer conductive for the ions starting from a specific temperature,
a first electrode and a second electrode spaced apart from one another by the transport layer,
a heating device for heating the transport layer to the specific temperature,
a first voltage source applying a control voltage to the heating device, and
a second voltage source applying a voltage difference across the first and second electrodes,
wherein the voltage difference between the first electrode and the second electrode provides a stream of ions from the first electrode through the transport layer to the second electrode if the first and second electrodes are in contact with the gas,
wherein the first voltage source provides a voltage potential of zero volts averaged over time or provides the voltage potential of the first electrode at the heating device.
